(19) United States
(12) Patent Application Publication
(10) Pub. No.: US 2023/0240754 A1
(43) Pub. Date: Aug. 3, 2023

(54) TISSUE PATHWAY CREATION USING ULTRASONIC SENSORS

(71) Applicant: Mazor Robotics Ltd., Caesarea (IL)

(72) Inventors: Elad Rotman, Netanya (IL); Eitan Detinis, Shefayim (IL); Noam Weiss, Haifa (IL); Adi Ess, Ramat Gan (IL); Ido Zucker, Tel Aviv (IL)

(21) Appl. No.: 17/591,004

(22) Filed: Feb. 2, 2022

(51) Int. Cl. A61B 34/20; A61B 34/32; A61B 8/00; A61B 17/32

(52) U.S. Cl. CPC A61B 34/20; A61B 34/32; A61B 8/461; A61B 8/4218; A61B 17/32; A61B 2034/2063

(57) ABSTRACT

A robotic surgical system according to at least one embodiment of the present disclosure includes a first robot arm coupled to a second robot arm, where a surgical tool is attached to the first robot arm and an ultrasonic sensor is attached to the second robot arm. Accordingly, when the first robot arm is moved to position the surgical tool adjacent a target site of a patient, the second robot arm is also moved to position the ultrasonic sensor adjacent the surgical tool and the target site. In some examples, images generated by the ultrasonic sensor may be used to determine layers of fascia of the patient at the target site. Subsequently, the surgical tool may move through the layers of fascia while continuing to receive real-time images of the tissue displacement instrument relative to the target site from the ultrasonic sensor.

### BACKGROUND

The present disclosure is generally directed to surgical systems and relates more particularly to robotic surgical devices.

Surgical robots may assist a surgeon or other medical provider in carrying out a surgical procedure or may complete one or more surgical procedures autonomously. Providing controllable linked articulating members allows a surgical robot to reach areas of a patient anatomy during various medical procedures.

### BRIEF SUMMARY

Example aspects of the present disclosure include:

A robotic surgical system, comprising: a first robot arm comprising a proximal end and a distal end; a surgical tool attached to the distal end of the first robot arm, the surgical tool comprising a tissue displacement instrument; an ultrasonic sensor; a processor coupled with the first robot arm and the ultrasonic sensor; and a memory coupled with and readable by the processor and storing therein data that, when executed by the processor, cause the processor to: move the first robot arm into a first pose positioning the tissue displacement instrument adjacent a target site of a patient; move the ultrasonic sensor into a second pose such that the ultrasonic sensor is aimed at the tissue displacement instrument and the target site; receive a first image from the ultrasonic sensor of the tissue displacement instrument relative to the target site; determine, based on the first image, layers of fascia of the patient at the target site, the layers of fascia being disposed between an outer layer of skin and an internal point of the target site; move, by actuating the first robot arm, the tissue displacement instrument through the layers of the fascia while continuing to receive real-time images of the tissue displacement instrument relative to the target site; and move the ultrasonic sensor such that the ultrasonic sensor is continually aimed at the tissue displacement instrument and the target site as the tissue displacement instrument is moved.

Any of the aspects herein, wherein the tissue displacement instrument comprises: a first end comprising a blade; and a second end comprising a blunt end, wherein the surgical tool is rotatable along an axis disposed between the first end and the second end, wherein, in a first tool position, the first end is disposed adjacent the target site, and wherein, in a second tool position, the surgical tool is rotated about the axis disposing the second end adjacent the target site in place of the first end.

Any of the aspects herein, wherein the data further cause the processor to determine, based on the first image, an orientation of the layers of fascia; and rotate, based on the orientation of the layers of fascia, between the first tool position for cutting fascia to the second tool position for displacing fascia.

Any of the aspects herein, wherein the data further cause the processor to receive a second image from the ultrasonic sensor of the tissue displacement instrument as the tissue displacement instrument is moved; and determine, based on the second image, one or more characteristics of the layers of fascia being disposed between the outer layer of skin and the internal point of the target site.

Any of the aspects herein, wherein the data further cause the processor to stop movement of the tissue displacement instrument and the ultrasonic sensor based on the one or more characteristics.

Any of the aspects herein, wherein the one or more characteristics comprise a physical measurement of the layers of fascia, an indication that the layers of fascia are fully breached, or a combination thereof.

Any of the aspects herein, wherein the data further cause the processor to move the tissue displacement instrument and the ultrasonic sensor based on the one or more characteristics.

Any of the aspects herein, wherein the data to move the tissue displacement instrument and the ultrasonic sensor further cause the processor to rotate the tissue displacement tool to a first tool position for cutting fascia from a second tool position for displacing fascia based on the one or more characteristics.

Any of the aspects herein, wherein the one or more characteristics comprise a physical measurement of the layers of fascia, an indication that one or more layers of the layers of fascia are yet to be breached, or a combination thereof.

Any of the aspects herein, further comprising: a display screen coupled to the ultrasonic sensor, wherein the display screen receives and displays the real-time images from the ultrasonic sensor of the tissue displacement instrument relative to the target site.

Any of the aspects herein, wherein the real-time images received from the ultrasonic sensor of the tissue displacement instrument comprise ultrasound images.

Any of the aspects herein, wherein the ultrasonic sensor comprises a transducer.

Any of the aspects herein, further comprising: a second robot arm comprising a proximal end and a distal end, wherein the ultrasonic sensor is attached to the distal end of the second robot arm.

A surgical robot, comprising: a first robot arm comprising a proximal end and a distal end; a surgical tool attached to the distal end of the first robot arm, the surgical tool comprising a tissue displacement instrument; and an ultrasonic sensor, wherein the ultrasonic sensor is coupled to the first robot arm such that the ultrasonic sensor is continually aimed at the tissue displacement instrument and a target site as the tissue displacement instrument is moved.

Any of the aspects herein, wherein the tissue displacement instrument comprises: a first end comprising a blade; and a second end comprising a blunt end, wherein the surgical tool is rotatable along an axis disposed between the first end and the second end, wherein, in a first tool position, the first end is disposed adjacent the target site, and wherein, in a second tool position, the surgical tool is rotated about the axis disposing the second end adjacent the target site in place of the first end.

Any of the aspects herein, wherein the tissue displacement instrument is rotated between the first tool position for cutting fascia and the second tool position for displacing fascia based on a first image generated by the ultrasonic sensor.

Any of the aspects herein, wherein the first image generated by the ultrasonic sensor indicates an orientation of layers of fascia.

Any of the aspects herein, further comprising: a display screen coupled to the ultrasonic sensor, wherein the display screen receives and displays real-time images from the ultrasonic sensor of the tissue displacement instrument relative to the target site.

Any of the aspects herein, wherein the real-time images from the ultrasonic sensor comprise ultrasound images.

A system, comprising: a processor coupled with a first robot arm and an ultrasonic sensor; and a memory coupled with and readable by the processor and storing therein data that, when executed by the processor, cause the processor to: move the first robot arm into a first pose positioning a tissue displacement instrument adjacent a target site of a patient; move the ultrasonic sensor into a second pose such that the ultrasonic sensor is aimed at the tissue displacement instrument and the target site; receive a first image from the ultrasonic sensor of the tissue displacement instrument relative to the target site; determine, based on the first image, layers of fascia of the patient at the target site, the layers of fascia being disposed between an outer layer of skin and an internal point of the target site; move, by actuating the first robot arm, the tissue displacement instrument through the layers of the fascia while continuing to receive real-time images of the tissue displacement instrument relative to the target site; and move the ultrasonic sensor such that the ultrasonic sensor is continually aimed at the tissue displacement instrument and the target site as the tissue displacement instrument is moved.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a block diagram of a system according to at least one embodiment of the present disclosure;

FIG. 2A is a perspective diagram of a robotic surgical system according to at least one embodiment of the present disclosure;

FIG. 2B is a perspective diagram of a robotic surgical system according to at least one embodiment of the present disclosure;

FIG. 3A is a perspective diagram of a surgical tool according to at least one embodiment of the present disclosure;

FIG. 3B is a side view diagram of the surgical tool according to at least one embodiment of the present disclosure;

FIG. 3C is a top view diagram of the surgical tool according to at least one embodiment of the present disclosure;

FIG. 3D is a bottom view diagram of the surgical tool according to at least one embodiment of the present disclosure;

FIG. 4A is a block diagram of a robotic surgical system according to at least one embodiment of the present disclosure;

FIG. 4B is a block diagram of the robotic surgical system according to at least one embodiment of the present disclosure;

FIGS. 5-6 are flowcharts of a method for operating a robotic surgical system according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia GeForce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

During minimal invasive surgeries (e.g., minimal invasive spinal surgeries), a physician or surgeon may be unable to see a specific area to be reached through the body (e.g., a selected vertebra section) that is a target for the minimal invasive surgeries. Additionally, creating tissue pathways for the minimal invasive surgeries can be a difficult task without being able to see internal areas of the patient. For example, without viewing fascia (e.g., or other tissue layers) using the human eye, the surgeon cannot be sure if they have reached fascia, passed the fascia, or stressed the fascia until the spine bone level is reached.

As described herein, ultrasound imaging can be used on a planned trajectory for creating a tissue pathway to extrude information to assist in a surgery, such as a physical measurement of fascia between a given tool and a target site. For example, a robotic surgical system may include a first robot arm with a surgical tool attached to a distal end of the first robot arm, where the surgical tool includes a tissue displacement instrument, and the robotic surgical system may also include an ultrasonic sensor (e.g., transducer). The first robot arm and the ultrasonic sensor may be coupled together (e.g., via a processor), such that if the first robot arm is moved, the ultrasonic sensor is also moved to position the ultrasonic sensor to be aimed at the surgical tool of the first robot arm. Accordingly, the first robot arm and the attached surgical tool can be moved through layers of tissue (e.g., layers of fascia) while continuing to receive real-time images from the ultrasonic sensor to assist with the surgery. In some examples, the ultrasonic sensor may be attached to a distal end of a second robot arm, where the first robot arm and the second robot arm are coupled together (e.g., via a processor).

In some examples, the tissue displacement instrument of the surgical tool attached to the first robot arm may include a first end that has a blade and a second end that has a blunt end. Additionally, the surgical tool may be rotatable along an axis (e.g., a tool rotation axis) disposed between the first end and the second end, such that the surgical tool can be positioned to present the first end adjacent to a target site or to present the second end adjacent to the target site. That is, the surgical tool can be placed in a first tool position where the first end and the blade are disposed adjacent to the target site and can also be rotated about the axis to be placed into a second tool position where the second end and the blunt end are disposed adjacent the target site. Subsequently, when using this surgical tool that is a combination cutting and tissue pathway creation tool in tandem with the ultrasonic sensor, the robotic surgical system may determine an orientation of layers of fascia based on real-time images (e.g., ultrasound images) from the ultrasonic sensor and may rotate the surgical tool between the first tool position and the second tool position based on the orientation of layers of fascia.

For example, while using the surgical tool in the second tool position for displacing fascia with the blunt end, if the real-time images indicate that there are layers of fascia between the blunt end and a target site (e.g., one or more layers of fascia yet to be breached), the surgical tool may be rotated (e.g., autonomously or by a user operating the robotic surgical system) about the axis to the first tool position for cutting fascia with the blade. Additionally or alternatively, based on the real-time images received from the ultrasonic sensor, the robotic surgical system and/or a user operating the robotic surgical system may determine how much force (e.g., an amount of pressure) to apply with the blade or the blunt end to breach the fascia or other tissue layers. Once the layers of fascia are breached (e.g., as shown with the real-time images from the ultrasonic sensor), the surgical tool may be rotated about the axis back to the second tool position to displace the fascia and create a tissue pathway and/or dilation using the blunt end. Additionally or alternatively, if the real-time images indicate that the layers of fascia have been fully breached, the robotic surgical system and/or a user operating the robotic surgical system may stop movement of the surgical tool and the ultrasonic sensor. In some examples, a display screen may be coupled to the ultrasonic sensor to receive and display these real-time images from the ultrasonic sensor.

Embodiments of the present disclosure provide technical solutions to one or more of the problems of (1) performing a surgery where a surgeon is unable to see the specific area to be reached through the body, (2) creating tissue pathways without being able to see internal areas of the patient, and (3) unnecessarily prolonged surgeries. For example, a robotic surgical system and a method of utilizing an ultrasonic sensor, or transducer, in combination with a multi-ended surgical tool (e.g., having a blade end and a blunt end) are provided that allows a proper tool to be selected depending on movement through fascia. In one example, as the surgical tool passes through various layers of fascia, images from the ultrasonic sensor (e.g., showing internal areas to be reached) may indicate that a portion of the fascia may need to be cut rather than displaced, to reduce pressure, force applied, and/or risk to the patient. In this example, the surgical tool may be rotated from arranging one end adjacent to the target site to arranging the other end adjacent to the target site, and vice versa. Compared to traditional procedures, the procedure described herein allows a greater number of operations to be performed in a shorter amount of times, which results in lower risks to a patient, shorter operative times, lower operation costs, and enhanced patient recovery.

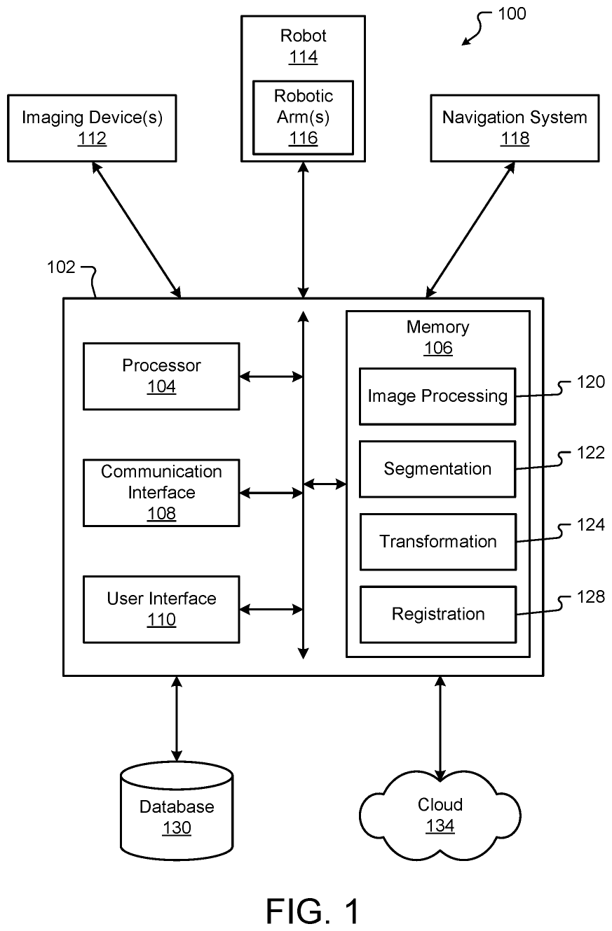

Turning first to FIG. 1, a block diagram of a system 100 according to at least one embodiment of the present disclosure is shown. The system 100 may be used to operate a robot 114 and multiple robotic arms 116 of the robot 114, where a first robotic arm 116 includes an attached surgical tool that includes both a blade for cutting different tissue layers (e.g., skin, fat, fascia, etc.) and a blunt tip trocar with a dilator for creating a highly accurate pathway through the tissue layers to a target site of a patient (e.g., a selected vertebra) and a second robotic arm 116 includes an attached ultrasonic sensor (e.g., an imaging device 112). The first robotic arm 116 and the second robotic arm 116 may be coupled together (e.g., via a processor 104), such that if the first robotic arm 116 is moved, the second robotic arm 116 is moved to position the ultrasonic sensor of the second robotic arm 116 adjacent to the surgical tool of the first robotic arm 116. Accordingly, the first robotic arm 116 and the attached surgical tool can be moved through layers of tissue (e.g., layers of fascia) while continuing to receive real-time images from the ultrasonic sensor, which can provide information on which part of the surgical tool (e.g., blade or blunt tip trocar) should be used or an amount of pressure to apply with the surgical tool. Additionally or alternatively, the ultrasonic sensor may not be attached to the second robotic arm 116 and may be moved as its own component such that the ultrasonic sensor is continually aimed at the surgical tool (e.g., attached to the first robotic arm 116) and the target site of the patient.

In some examples, the system 100 may control, pose, and/or otherwise manipulate a surgical mount system, a surgical arm, and/or surgical tools attached thereto and/or carry out one or more other aspects of one or more of the methods disclosed herein. The system 100 comprises a computing device 102, one or more imaging devices 112, a robot 114, a navigation system 118, a database 130, and/or a cloud or other network 134. Systems according to other embodiments of the present disclosure may comprise more or fewer components than the system 100. For example, the system 100 may not include the imaging device 112, the robot 114, the navigation system 118, one or more components of the computing device 102, the database 130, and/or the cloud 134.

The computing device 102 comprises a processor 104, a memory 106, a communication interface 108, and a user interface 110. Computing devices according to other embodiments of the present disclosure may comprise more or fewer components than the computing device 102.

The processor 104 of the computing device 102 may be any processor described herein or any similar processor. The processor 104 may be configured to execute instructions stored in the memory 106, which instructions may cause the processor 104 to carry out one or more computing steps utilizing or based on data received from the imaging device 112, the robot 114, the navigation system 118, the database 130, and/or the cloud 134.

The memory 106 may be or may comprise RAM, DRAM, SDRAM, other solid-state memory, any memory described herein, or any other tangible, non-transitory memory for storing computer-readable data and/or instructions. The memory 106 may store information or data useful for completing, for example, any step of the methods 500 and/or 600 described herein, or of any other methods. The memory 106 may store, for example, instructions and/or machine learning models that support one or more functions of the robot 114. For instance, the memory 106 may store content (e.g., instructions and/or machine learning models) that, when executed by the processor 104, enable image processing 120, segmentation 122, transformation 124, and/or registration 128. Such content, if provided as in instruction, may, in some embodiments, be organized into one or more applications, modules, packages, layers, or engines. Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the imaging device 112, the robot 114, the database 130, and/or the cloud 134.

The computing device 102 may also comprise a communication interface 108. The communication interface 108 may be used for receiving image data or other information from an external source (such as the imaging device 112, the robot 114, the navigation system 118, the database 130, the cloud 134, and/or any other system or component not part of the system 100), and/or for transmitting instructions, images, or other information to an external system or device (e.g., another computing device 102, the imaging device 112, the robot 114, the navigation system 118, the database 130, the cloud 134, and/or any other system or component not part of the system 100). The communication interface 108 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 108 may be useful for enabling the device 102 to communicate with one or more other processors 104 or computing devices 102, whether to reduce the time needed to accomplish a computing-intensive task or for any other reason.

The computing device 102 may also comprise one or more user interfaces 110. The user interface 110 may be or may comprise a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some embodiments, the user interface 110 may be useful to allow a surgeon or other user to modify instructions to be executed by the processor 104 according to one or more embodiments of the present disclosure, and/or to modify or adjust a setting of other information displayed on the user interface 110 or corresponding thereto.

Although the user interface 110 is shown as part of the computing device 102, in some embodiments, the computing device 102 may utilize a user interface 110 that is housed separately from one or more remaining components of the computing device 102. In some embodiments, the user interface 110 may be located proximate to one or more other components of the computing device 102, while in other embodiments, the user interface 110 may be located remotely from one or more other components of the computer device 102.

The imaging device 112 may be operable to image anatomical feature(s) (e.g., a bone, veins, tissue, etc.) and/or other aspects of patient anatomy to yield image data (e.g., image data depicting or corresponding to a bone, veins, tissue, etc.). “Image data” as used herein refers to the data generated or captured by an imaging device 112, including in a machine-readable form, a graphical/visual form, and in any other form. In various examples, the image data may comprise data corresponding to an anatomical feature of a patient, or to a portion thereof. The image data may be or comprise a preoperative image, an intraoperative image, a postoperative image, or an image taken independently of any surgical procedure. In some embodiments, a first imaging device 112 may be used to obtain first image data (e.g., a first image) at a first time, and a second imaging device 112 may be used to obtain second image data (e.g., a second image) at a second time after the first time. The imaging device 112 may be capable of taking a 2D image or a 3D image to yield the image data. The imaging device 112 may be or comprise, for example, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MM) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other imaging device 112 suitable for obtaining images of an anatomical feature of a patient. The imaging device 112 may be contained entirely within a single housing or may comprise a transmitter/emitter and a receiver/detector that are in separate housings or are otherwise physically separated.

In some embodiments, the imaging device 112 may comprise more than one imaging device 112. For example, a first imaging device may provide first image data and/or a first image, and a second imaging device may provide second image data and/or a second image. In still other embodiments, the same imaging device may be used to provide both the first image data and the second image data, and/or any other image data described herein. The imaging device 112 may be operable to generate a stream of image data. For example, the imaging device 112 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

The robot 114 may be any surgical robot or surgical robotic system. The robot 114 may be or may comprise, for example, the Mazor X™ Stealth Edition robotic guidance system. The robot 114 may be configured to position the imaging device 112 at one or more precise position(s) and orientation(s), and/or to return the imaging device 112 to the same position(s) and orientation(s) at a later point in time. The robot 114 may additionally or alternatively be configured to manipulate a surgical tool (whether based on guidance from the navigation system 118 or not) to accomplish or to assist with a surgical task. In some embodiments, the robot 114 may be configured to hold and/or manipulate an anatomical element during or in connection with a surgical procedure. The robot 114 may comprise one or more robotic arms 116. In some embodiments, the robotic arm 116 may comprise a first robotic arm and a second robotic arm, though the robot 114 may comprise more than two robotic arms. In some embodiments, one or more of the robotic arms 116 may be used to hold and/or maneuver the imaging device 112. In embodiments where the imaging device 112 comprises two or more physically separate components (e.g., a transmitter and receiver), one robotic arm 116 may hold one such component, and another robotic arm 116 may hold another such component. Each robotic arm 116 may be positionable independently of the other robotic arm. The robotic arms 116 may be controlled in a single, shared coordinate space, or in separate coordinate spaces.

The robot 114, together with the robotic arm 116, may have, for example, one, two, three, four, five, six, seven, or more degrees of freedom. Further, the robotic arm 116 may be positioned or positionable in any pose, plane, and/or focal point. The pose includes a position and an orientation. As a result, an imaging device 112, surgical tool, or other object held by the robot 114 (or, more specifically, by the robotic arm 116) may be precisely positionable in one or more needed and specific positions and orientations.

The robotic arm(s) 116 may comprise one or more sensors that enable the processor 104 (or a processor of the robot 114) to determine a precise pose in space of the robotic arm (as well as any object or element held by or secured to the robotic arm).

In some embodiments, reference markers (e.g., navigation markers) may be placed on the robot 114 (including, e.g., on the robotic arm 116), the imaging device 112, or any other object in the surgical space. The reference markers may be tracked by the navigation system 118, and the results of the tracking may be used by the robot 114 and/or by an operator of the system 100 or any component thereof. In some embodiments, the navigation system 118 can be used to track other components of the system (e.g., imaging device 112) and the system can operate without the use of the robot 114 (e.g., with the surgeon manually manipulating the imaging device 112 and/or one or more surgical tools, based on information and/or instructions generated by the navigation system 118, for example).

The navigation system 118 may provide navigation for a surgeon and/or a surgical robot during an operation. The navigation system 118 may be any now-known or future-developed navigation system, including, for example, the Medtronic StealthStation™ S8 surgical navigation system or any successor thereof. The navigation system 118 may include one or more cameras or other sensor(s) for tracking one or more reference markers, navigated trackers, or other objects within the operating room or other room in which some or all of the system 100 is located. The one or more cameras may be optical cameras, infrared cameras, or other cameras. In some embodiments, the navigation system 118 may comprise one or more electromagnetic sensors. In various embodiments, the navigation system 118 may be used to track a position and orientation (e.g., a pose) of the imaging device 112, the robot 114 and/or the robotic arm 116, and/or one or more surgical tools (or, more particularly, to track a pose of a navigated tracker attached, directly or indirectly, in fixed relation to the one or more of the foregoing). The navigation system 118 may include a display for displaying one or more images from an external source (e.g., the computing device 102, imaging device 112, or other source) or for displaying an image and/or video stream from the one or more cameras or other sensors of the navigation system 118. In some embodiments, the system 100 can operate without the use of the navigation system 118. The navigation system 118 may be configured to provide guidance to a surgeon or other user of the system 100 or a component thereof, to the robot 114, or to any other element of the system 100 regarding, for example, a pose of one or more anatomical elements, whether or not a tool is in the proper trajectory, and/or how to move a tool into the proper trajectory to carry out a surgical task according to a preoperative or other surgical plan.

The database 130 may store information that correlates one coordinate system to another (e.g., one or more robotic coordinate systems to a patient coordinate system and/or to a navigation coordinate system). The database 130 may additionally or alternatively store, for example, one or more surgical plans (including, e.g., pose information about a target and/or image information about a patient's anatomy at and/or proximate the surgical site, for use by the robot 114, the navigation system 118, and/or a user of the computing device 102 or of the system 100); one or more images useful in connection with a surgery to be completed by or with the assistance of one or more other components of the system 100; and/or any other useful information. The database 130 may be configured to provide any such information to the computing device 102 or to any other device of the system 100 or external to the system 100, whether directly or via the cloud 134. In some embodiments, the database 130 may be or may comprise part of a hospital image storage system, such as a picture archiving and communication system (PACS), a health information system (HIS), and/or another system for collecting, storing, managing, and/or transmitting electronic medical records including image data.

The cloud 134 may be or represent the Internet or any other wide area network. The computing device 102 may be connected to the cloud 134 via the communication interface 108, using a wired connection, a wireless connection, or both. In some embodiments, the computing device 102 may communicate with the database 130 and/or an external device (e.g., a computing device) via the cloud 134.

The system 100 or similar systems may be used, for example, to carry out one or more aspects of any of the methods 500 and/or 600 described herein. The system 100 or similar systems may also be used for other purposes.

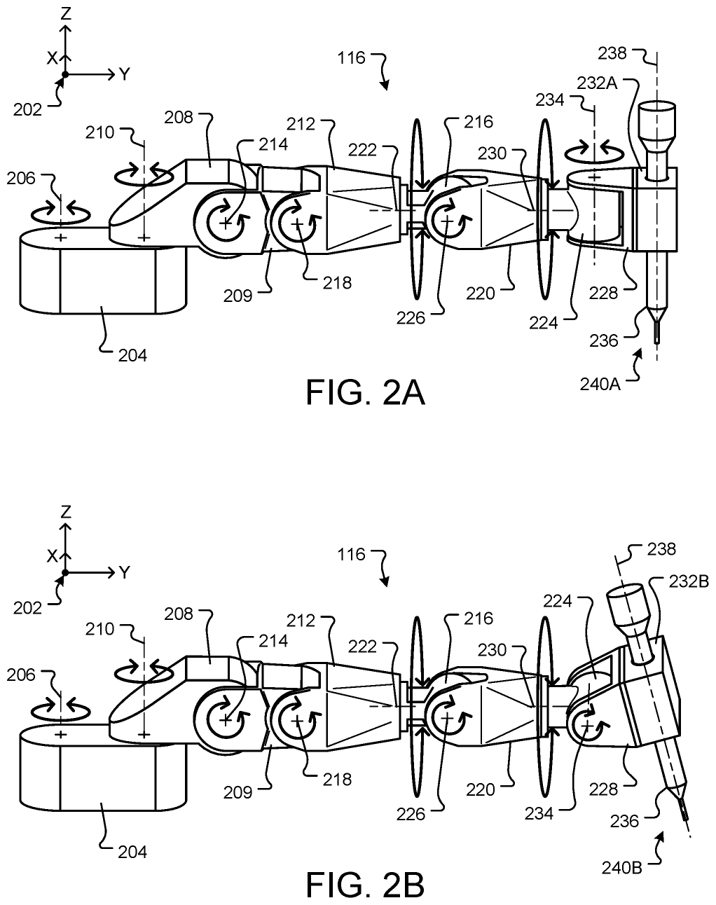

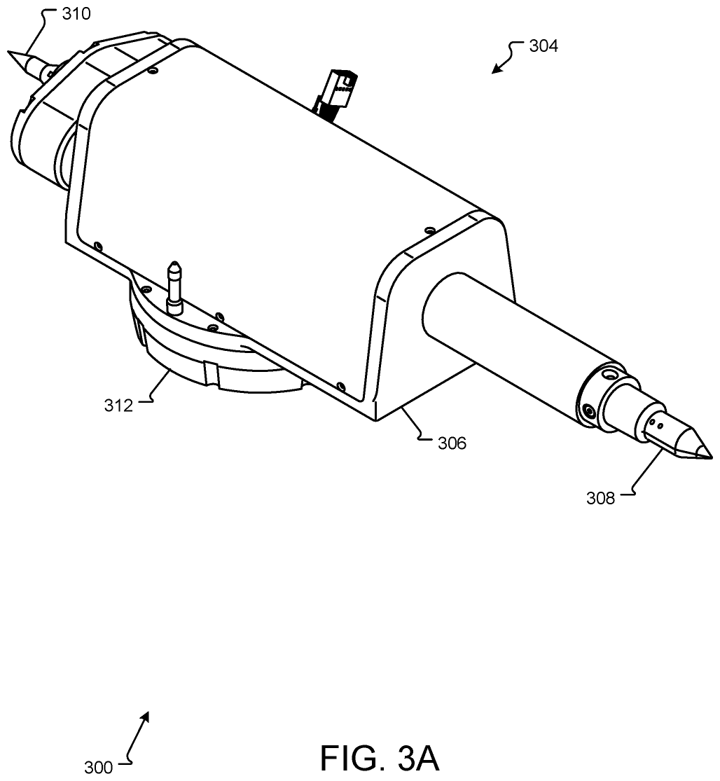

Referring now to FIGS. 2A and 2B, perspective diagrams of a robotic surgical system with different end effector 240A, 240B mount positions are shown in accordance with examples of the present disclosure. More specifically, FIGS. 2A and 2B show the robotic arm 116 of the robot 114 connected to an end effector 240A, 240B holding a surgical tool 236. As described herein, the surgical tool 236 may be a single surgical tool capable of cutting different tissue layers (e.g., skin, fat, fascia, etc.) using a blade end of the surgical tool 236 (e.g., a first end) and of creating a highly accurate pathway through the tissue layers to a target site of a patient (e.g., a selected vertebra) using a rod with a blunt tip end of the surgical tool 236 (e.g., a blunt end trocar with a dilator), where the surgical tool 236 is rotatable around a rotational axis to position an appropriate end of the surgical tool 236 to perform a corresponding action (e.g., for cutting or for creating a tissue pathway and/or dilation).

Additionally or alternatively, the surgical tool 236 may correspond to different surgical tools used between operations in a surgical application. For instance, a first surgical tool 236 may include a direction-specific blade that may require a specific rotational alignment and placement in the tool block 232A, 232B, while another surgical tool 236 may include a unidirectional cutting tool that is independent of rotational alignment in the tool block 232A, 232B.

Features of the robot 114 and/or robotic arm 116 may be described in conjunction with a coordinate system 202. The coordinate system 202, as shown in FIGS. 2A and 2B, includes three-dimensions comprising an X-axis, a Y-axis, and a Z-axis. Additionally or alternatively, the coordinate system 202 may be used to define planes (e.g., the XY-plane, the XZ-plane, and the YZ-plane) of the robot 114 and/or robotic arm 116. These planes may be disposed orthogonal, or at 90 degrees, to one another. While the origin of the coordinate system 202 may be placed at any point on or near the components of the robot 114, for the purposes of description, the axes of the coordinate system 202 are always disposed along the same directions from figure to figure, whether the coordinate system 202 is shown or not. In some examples, reference may be made to dimensions, angles, directions, relative positions, and/or movements associated with one or more components of the robot 114 and/or robotic arm 116 with respect to the coordinate system 202. For example, the width of the robotic arm 116 (e.g., running from the side shown in the foreground to the side in the background, into the page) may be defined as a dimension along the X-axis of the coordinate system 202, the height of the robotic arm 116 may be defined as a dimension along the Z-axis of the coordinate system 202, and the length of the robotic arm 116 (e.g., running from a proximal end at the first link 204 to a distal end at the seventh link 224, etc.) may be defined as a dimension along the Y-axis of the coordinate system 202. Additionally or alternatively, the height of the system 100 may be defined as a dimension along the Z-axis of the coordinate system 202, a reach of the robotic arm 116 may be defined as a dimension along the Y-axis of the coordinate system 202, and a working area of the robotic arm 116 may be defined in the XY-plane with reference to the corresponding axes of the coordinate system 202.

The robotic arm 116 may be comprised of a number of links 204, 208, 209, 212, 216, 220, 224 that interconnect with one another at respective axes of rotation 206, 210, 214, 218, 222, 226, 230, 234, or joints. There may be more or fewer links 204, 208, 209, 212, 216, 220, 224 and/or axes of rotation 206, 210, 214, 218, 222, 226, 230, 234 than are shown in FIGS. 2A and 2B. In any event, the robotic arm 116 may have a first link 204 disposed at a proximal end of the robotic arm 116 and an end mount flange 228 disposed furthest from the proximal end at a distal end of the robotic arm 116. The first link 204 may correspond to a base of the robotic arm 116. In some examples, the first link 204 may rotate about first rotation axis 206. A second link 208 may be connected to the first link 204 at a second rotation axis 210, or joint. The second link 208 may rotate about the second rotation axis 210. In one example, the first rotation axis 206 and the second rotation axis 210 may be arranged parallel to one another. For instance, the first rotation axis 206 and the second rotation axis 210 are shown extending along the Z-axis in a direction perpendicular to the XY-plane.

The robotic arm 116 may comprise a third link 209 that is rotationally interconnected to the second link 208 via the third rotation axis 214, or joint. The third rotation axis 214 is shown extending along the X-axis, or perpendicular to the first rotation axis 206 and second rotation axis 210. In this position, when the third link 209 is caused to move (e.g., rotate relative to the second link 208), the third link 209 (and the components of the robotic arm 116 extending from the third link 209) may be caused to move into or out of the XY-plane. The fourth link 212 is shown rotationally interconnected to the third link 209 via the fourth rotation axis 218, or joint. The fourth rotation axis 218 is arranged parallel to the third rotation axis 214. The fourth rotation axis 218 extends along the X-axis allowing rotation of the fourth link 212 into and out of the XY-plane.

In some examples, the robotic arm 116 may comprise one or more wrists 216, 224. The fifth link 216, or wrist, is shown rotationally interconnected to the fourth link 212 via a fifth rotation axis 222, or wrist joint. The fifth rotation axis 222 is shown extending along the Y-axis, which is perpendicular to the X-axis and the Z-axis. During operation of the robot 114, causing the fifth link 216 to rotate about the fifth rotation axis 222 may cause the components of the robotic arm 116 distal the joint at the fifth rotation axis 222 (e.g., the fifth link 216, the sixth link 220, the seventh link 224, the end mount flange 228, and the end effector 240A, 240B, etc.) to rotate about the Y-axis.

The sixth link 220 is rotationally interconnected to the fifth link 216 via the sixth rotation axis 226. The sixth rotation axis 226 extends along the X-axis and provides for rotation of the sixth link 220 relative to the fifth link 216 (e.g., into and out of the XY-plane in the position shown).

The seventh link 224, or wrist, is shown rotationally interconnected to the sixth link 220 via a seventh rotation axis 230, or wrist joint. The seventh rotation axis 230 is shown extending along the Y-axis (e.g., perpendicular to the X-axis and the Z-axis). During operation of the robot 114, causing the seventh link 224 to rotate about the seventh rotation axis 230 may cause the components of the robotic arm 116 distal the joint at the seventh rotation axis 230 (e.g., the end mount flange 228, and the end effector 240A, 240B, etc.) to rotate about the Y-axis.

Located at the distal end of the robotic arm 116, an end mount flange 228 may be rotationally interconnected to the end mount flange 228 via an eighth, or mount flange rotation, axis 234. In FIG. 2A, the seventh link 224 is positioned rotationally about the seventh rotation axis 230 such that the end mount flange 228 is oriented where the mount flange rotation axis 234 is extending along the Z-axis. In FIG. 2B, the seventh link 224 is positioned rotationally about the seventh rotation axis 230 such that the end mount flange 228 is oriented where the mount flange rotation axis 234 is extending along the X-axis. In some examples, at least the seventh link 224 may be rotated about the seventh rotation axis 230 to move between the end mount flange 228 position shown in FIG. 2A and the end mount flange 228 position shown in FIG. 2B, or vice versa. The end mount flange 228 and the mount flange rotation axis 234 may be the last movable (e.g., motor actuated, etc.) link and joint of the robotic arm 116. Moving between these two positions of the end mount flange 228 allows a particular end effector 240A, 240B to be attached and manipulated, or operated, according to a corresponding movement profile (e.g., range and limits) or set of kinematic solutions for the robot 114 (e.g., the robotic arm 116 and the surgical tool 236, etc.).

FIG. 2A shows first movement kinematics for the robotic arm 116 when the first tool block 232A of the first end effector 240A disposes the surgical tool axis 238 parallel to the mount flange rotation axis 234. In the position shown in FIG. 2A, rotation into and/or out of the XY-plane between the seventh link 224 and the first end effector 240A is prevented. This position and arrangement may be ideal for applications (e.g., operations, procedures, etc.) where an end rotational position of the surgical tool 236 may need to be maintained for the robotic arm 116. For example, the surgical tool 236 in the first end effector 240A may correspond to an imaging device that may need to be maintained in a particular nonrotational position relative to a patient during imaging (e.g., where an imaging plane of the surgical tool 236 should be maintained parallel to the XY-plane as other joints of the robotic arm 116 move the distal end closer to or further from the proximal end). In this case, the corresponding arrangement of the surgical tool axis 238 (e.g., parallel to the mount flange rotation axis 234) associated with the first end effector 240A may be preferred. In another example, rotation of the surgical tool 236 into, or out of, the XY-plane may need to be prevented to ensure accuracy of movement along the Y-axis, in the XY-plane, and/or the like. Additionally or alternatively, a distance between a reference plane and an end of the surgical tool 236 (e.g., along the Z-axis) may need to remain constant during operation of the robot 114. In any of these cases, the position and arrangement shown in conjunction with FIG. 2A (e.g., preventing end rotation relative to the XY-plane) may be preferred.

FIG. 2B shows second movement kinematics for the robotic arm 116 when the second tool block 232B of the second end effector 240B disposes the surgical tool axis 238 perpendicular (e.g., at 90 degrees) to the mount flange rotation axis 234. In this alternative position, the end mount flange 228 and second end effector 240B may be allowed to rotate relative to the seventh link 224. Stated another way, in this alternative position, the end mount flange 228 and second end effector 240B may be allowed to rotate into and/or out of the XY-plane (e.g., relative to seventh link 224 at the mount flange rotation axis 234). This position and arrangement may be ideal when a precise rotational movement of the surgical tool 236 at the distal end of the robotic arm 116 is desired. In contrast to the position and arrangement shown in FIG. 2A, where the closest rotation of the first end effector 240A about the X-axis is provided at the sixth rotation axis 226, the position and arrangement of FIG. 2B allows the second end effector 240B to be rotated about the X-axis about the mount flange rotation axis 234. Among other things, this position and arrangement may be used for any application where a movement of the second end effector 240B including an end rotation into and/or out of the XY-plane is desired for the surgical tool 236. Such applications may include directional cutting operations, probing movements, displacement of tissue and organs, and/or other surgical operations.

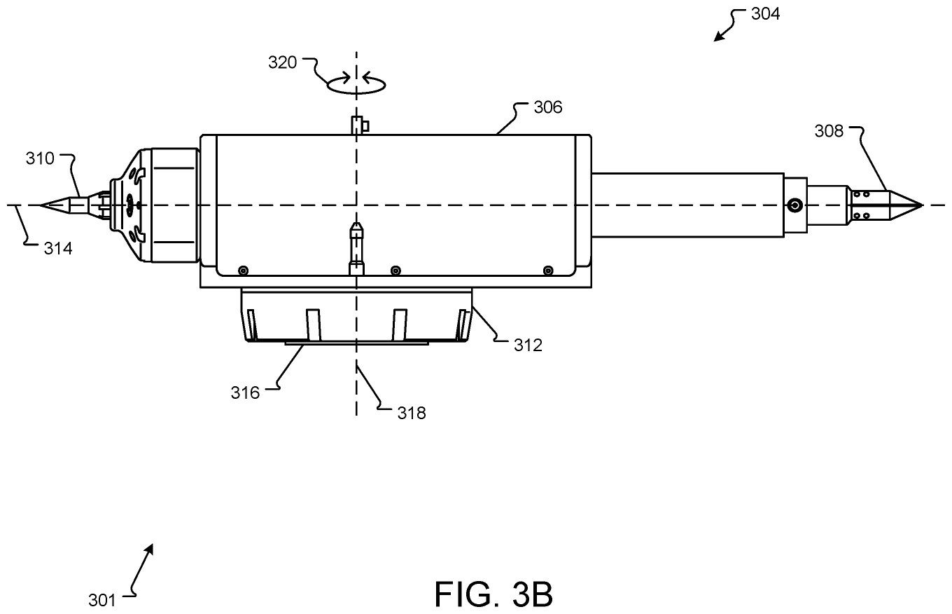

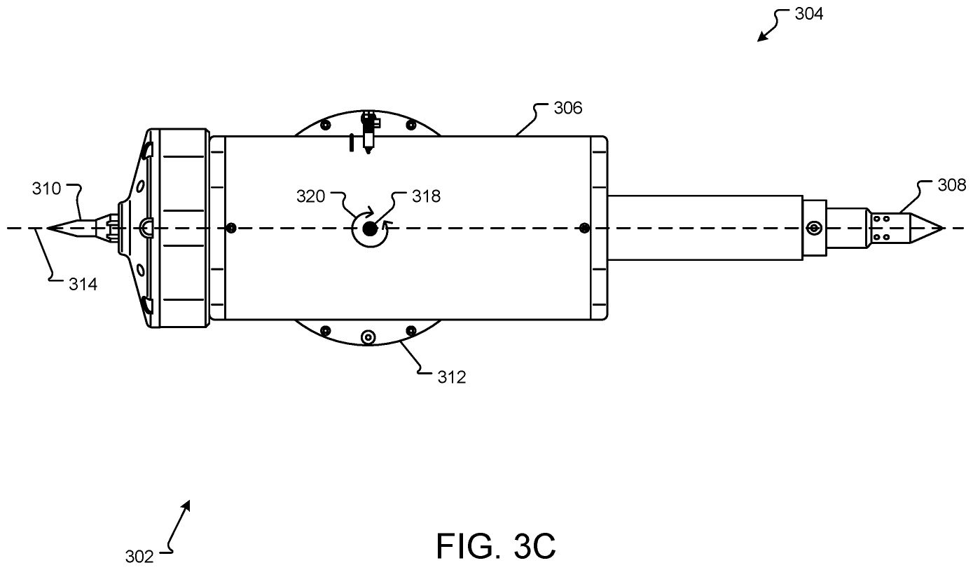

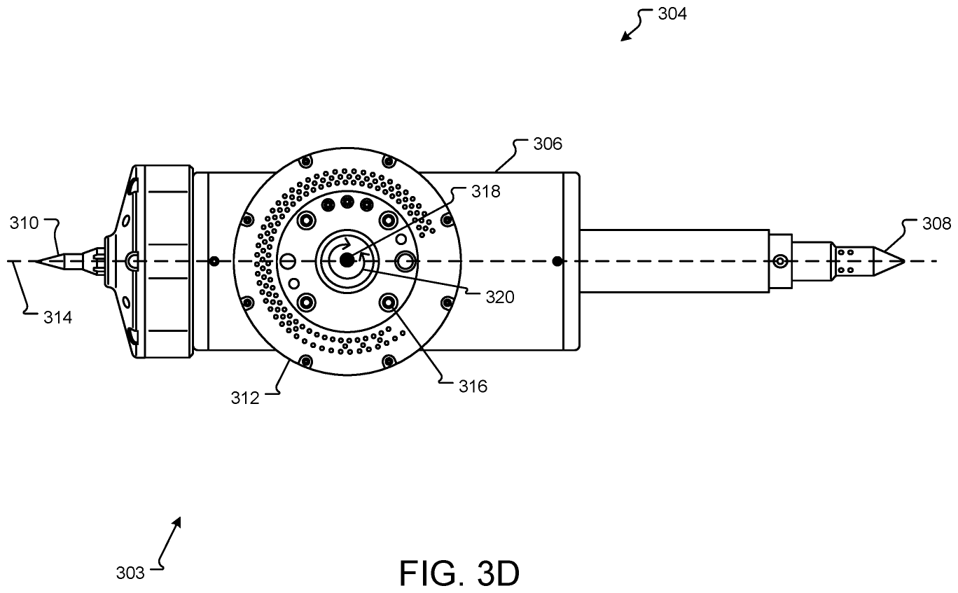

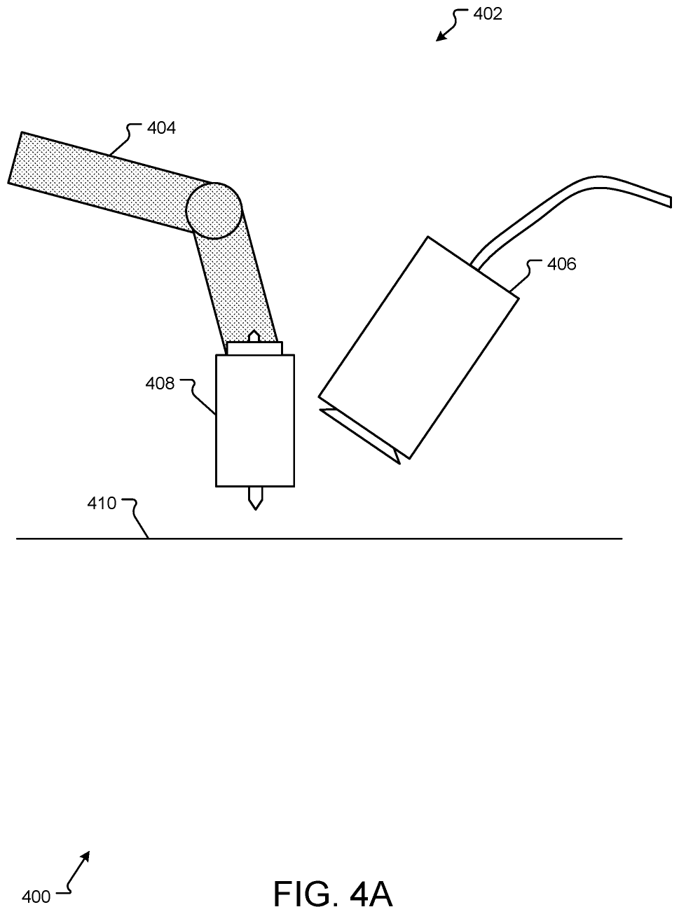

FIG. 3A shows a perspective diagram 300 of a surgical tool 304 according to at least one embodiment of the present disclosure. As illustrated in FIG. 3A, the surgical tool 304 may include a housing 306, a blade support tip 308 extending from a first end of the housing 306, a rod 310 (e.g., a trocar) that extends from a second end of the housing 306 in a direction away from the first end of the housing 306, and a robot interface bracket 312 that is coupled to the housing 306. The blade support tip 308 may include a blade that is disposed at least partially within the blade support tip 308, where the blade includes a sharpened edge. The blade may be moveable between a retracted stage where the sharpened edge is concealed within the blade support tip 308 and an extended state where the sharpened edge is exposed from the blade support tip 308. The rod 310 may include a blunt tip end and an actuation end, where the actuation end is disposed within the housing 306 and where the blunt tip end extends from the second end of the housing 306.

FIG. 3B shows a side view diagram 301 of the surgical tool 304 according to at least one embodiment of the present disclosure. As illustrated in FIG. 3B, the housing 306 may include a longitudinal axis 314 that extends from the first end of the housing 306 (e.g., from which the blade support tip 308 extends) to the second end of the housing 306 (e.g., from which the rod 310 extends). Accordingly, the blade support tip 308 extends from the first end of the housing 306 in a direction away from the second end of the housing 306 along the longitudinal axis 314, and the blunt tip end of the rod 310 extends from the second end of the housing 306 in a direction away from the first end of the housing 306 along the longitudinal axis 314.

Additionally, the robot interface bracket 312 coupled to the housing 306 may include a robot mount flange 316 that further includes a tool rotation axis 318 arranged perpendicular to the longitudinal axis 314. The surgical tool 304 can attach to a distal end of a robot arm (e.g., a robot arm 116 of a robot 114 or a robotic surgical system) via the robot mount flange 316 (e.g., by attaching to an end mount flange of the robot arm as described with reference to FIGS. 2A and 2B). In some examples, the surgical tool 304 may be able to perform a rotation 320 about the tool rotation axis 318 (e.g., the surgical tool 304 is rotatable). With the rotation 320, the surgical tool 304 can rotate between a cutting position disposing the blade support tip 308 in proximity to a target site and a tissue pathway creation position disposing the blunt tip end of the rod 310 in proximity to the target site. That is, the surgical tool 304 can rotate between presenting the blade support tip 308 at the target site (e.g., for cutting skin, fascia, fat, etc.) and presenting or extending the blunt tip end of the rod 310 at the target site (e.g., for tissue pathway creation and/or dilation creation) without having to change out the surgical tool 304 to perform individual steps of a surgical operation.

FIG. 3C shows a top view diagram 302 of the surgical tool 304 according to at least one embodiment of the present disclosure, and FIG. 3D shows a bottom view diagram 303 of the surgical tool 304 according to at least one embodiment of the present disclosure. As shown in FIG. 3D, the robot mount flange 316 on the robot interface bracket 312 may include one or more kinematic coupling dimples that enable attachment of the surgical tool 304 to a distal end of a robot arm (e.g., a robot arm 116 of a robot 114 or a robotic surgical system by attaching to an end mount flange of the robot arm as described with reference to FIGS. 2A and 2B).

As described previously, the surgical tool 304 may be rotatable around the tool rotation axis 318 between the cutting position (e.g., disposing the blade support tip 308 in proximity to the target site) and the tissue pathway creation position (e.g., disposing the blunt tip end of the rod 310 in proximity to the target site). For example, as a non-limiting surgical operation example, the surgical tool 304 may first rotate to the cutting position to present the blade support tip 308 in proximity to the target site for creating an incision in the skin of a patient. Subsequently, the surgical tool 304 may rotate to the tissue pathway creation position (e.g., about the tool rotation axis 318) to present the blunt tip end of the rod 310 in proximity to the target site to create and/or dilate a tissue pathway to reach an internal site of the patient corresponding to the target site. The surgical tool 304 may then rotate between the cutting position to cut through additional tissue layers (e.g., fascia, fat, etc.) as needed and the tissue pathway creation position when those additional tissue layers are punctured until the internal site is reached and the surgical operation is finished (e.g., a screw is inserted, a cage is inserted, a drill is inserted, or an additional minimal invasive surgery (MIS) tool is used/inserted).

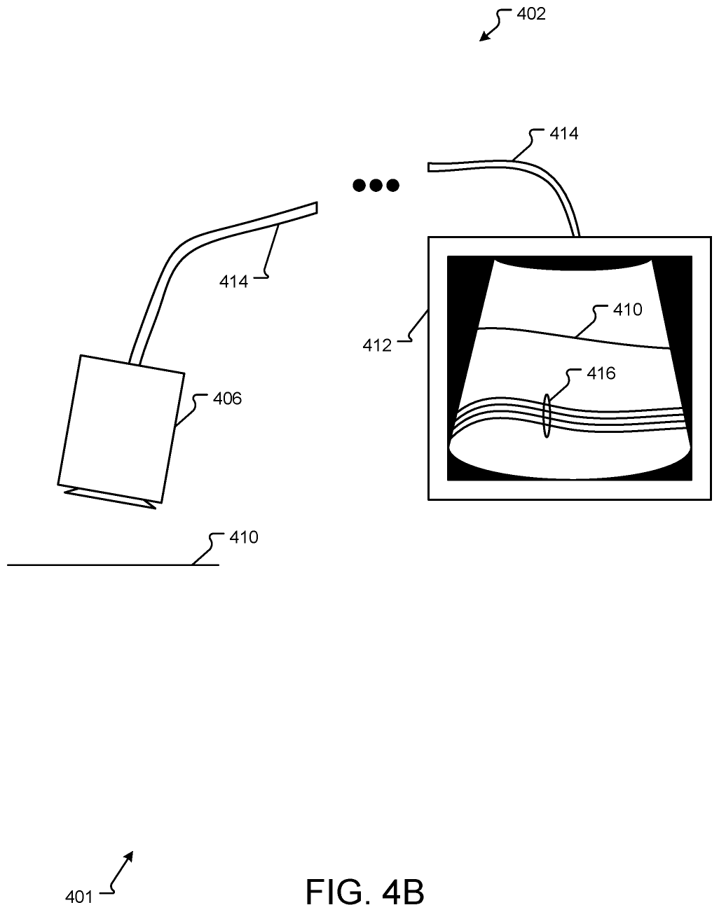

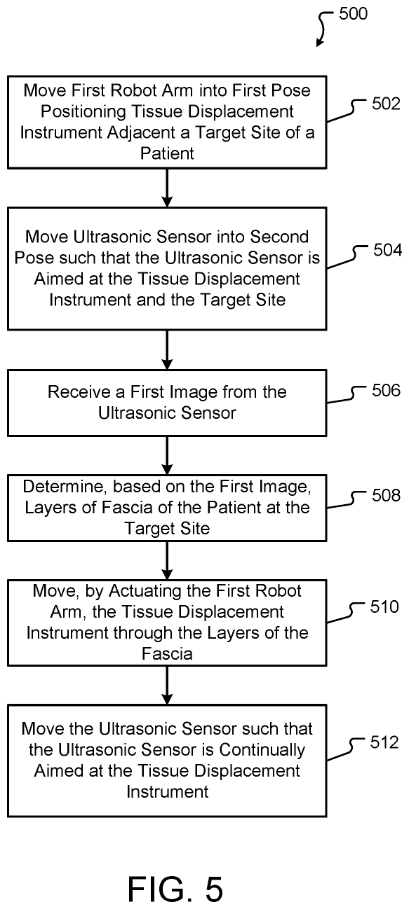

FIG. 4A shows a block diagram 400 of a robotic surgical system 402 according to at least one embodiment of the present disclosure. As illustrated in FIG. 4A and described herein, the robotic surgical system 402 may include a first robot arm 404 and an ultrasonic sensor 406 (e.g., the first robot arm 404 may be an example of the robotic arm(s) 116 as described with reference to FIGS. 1 and 2).

The first robot arm 404 may include a proximal end and a distal end, where a surgical tool 408 is attached to the distal end of the first robot arm 408. In some examples, the surgical tool 408 may be an example of the surgical tool 304 as described with reference to FIGS. 3A-3D. For example, the surgical tool 408 may include a first end and a second end, where the first end includes a blade (e.g., disposed at least partially within a blade support tip as described with reference to FIGS. 3A-3D) and the second end includes a blunt end (e.g., blunt tip end of a rod as described with reference to FIGS. 3A-3D). Additionally, the surgical tool 408 may be rotatable along an axis disposed between the first end and the second end (e.g., a tool rotation axis as described with reference to FIGS. 3A-3D). For example, the surgical tool 408 may be rotatable between at least a first tool position and a second tool position. The first tool position for the surgical tool 408 may include the first end (e.g., with the blade) being disposed adjacent a target site of a patient, and the second tool position for the surgical tool 408 may include the second end (e.g., with the blunt end) being disposed adjacent the target site. In some examples, the surgical tool 408 may be or may be referred to as a tissue displacement instrument.

In some examples, the ultrasonic sensor 406 may be or may be referred to as a transducer. The ultrasonic sensor 406 may be an example of an imaging device 112 as described with reference to FIG. 1. In some examples, the ultrasonic sensor 406 may capture images (e.g., ultrasound images) on a sagittal or longitudinal plane of a patient, where the images captured by the ultrasonic sensor 406 depict different parts of the patient, such as bone, soft tissue, fascia, metal, etc. Additionally or alternatively, although not shown, the robotic surgical system 402 may include a second robot arm that includes a proximal end and a distal end, where the ultrasonic sensor 406 is attached to the distal end of the second robot arm.

In some examples, the first robot arm 404 and the ultrasonic sensor 406 may be coupled together via a processor of the robotic surgical system 402 (e.g., a processor 104 as described with reference to FIG. 1). With the first robot arm 404 and the ultrasonic sensor 406 being coupled together, movements of both the first robot arm 404 and the ultrasonic sensor 406 may be synchronized. For example, if the first robot arm 404 is moved into a first pose positioning the surgical tool 408 adjacent a target site of a patient, the ultrasonic sensor 406 may be moved into a second pose such that the ultrasonic sensor 406 is aimed at the surgical tool 408 and the target site. In some examples, an angle of the ultrasonic sensor 406 may be moved as the surgical tool 408 moves (e.g., as one of the ends of the surgical tool 408 moves, such as the blunt end or the blade). Additionally, the second pose of the ultrasonic sensor 406 and/or a placement or position of the ultrasonic sensor 406 may be inferred from a detection of a location or position of the surgical tool 408 attached to the first robot arm 404 using a reference coordinate system. If the ultrasonic sensor 406 is attached to the distal end of a second robot arm, the position of the second robot arm may be inferred from the detection of the surgical tool 408 using the reference coordinate system.

Accordingly, the ultrasonic sensor 406 may provide images (e.g., of internal areas of the patient) to assist in operating the robotic surgical system 402 and moving the surgical tool 408 by having the movements of the corresponding robot arms synchronized. For example, the ultrasonic sensor 406 may generate a first image of the surgical tool 408 relative to the target site, where layers of fascia disposed between an outer layer of skin 410 and an internal point of the target site of the patient can be determined based on the first image. Subsequently, by actuating the first robot arm 404, the surgical tool 408 can be moved through the layers of the fascia while continuing to receive real-time images of the surgical tool 408 relative to the target site, where the ultrasonic sensor 406 is also moved (e.g., by actuating the second robot arm if the ultrasonic sensor 406 is attached to the distal end of the second robot arm) such that the ultrasonic sensor 406 is continually aimed at the surgical tool 408 and the target site as the surgical tool 408 is moved.

In some examples, based on the real-time images generated by the ultrasonic sensor 406, one or more characteristics of the layers of fascia can be determined, such as an orientation of the layers of fascia, a physical measurement of the layers of fascia, etc. Based on the one or more characteristics determined from the real-time images generated by the ultrasonic sensor 406, the surgical tool 408 may perform different actions or may be controlled to perform different actions. For example, based on the orientation of the layers of fascia, the surgical tool 408 may rotate between the first tool position for cutting fascia (e.g., with the blade) and the second tool position for displacing fascia (e.g., with the blunt end).

Additionally or alternatively, based on the physical measurement of the layers of fascia, the surgical tool 408 may rotate to the first tool position for cutting fascia or may stop moving. For example, if the physical measurement of fascia indicates that one or more of the layers of fascia have not been breached, the surgical tool 408 may rotate to the first tool position for cutting fascia to cut through the one or more unbreached layers of fascia before rotating back to the second tool position for displacing fascia and continuing to create a tissue pathway. Instead of applying more force or pressure when reaching an area of high resistance (e.g., such as fascia), the real-time images generated by the ultrasonic sensor 406 can indicate the presence of fascia, where the surgical tool 408 can then be rotated to the first tool position to use the blade to puncture then fascia and then rotate again to the second tool position for displacing fascia and continuing the surgical operation. In some examples, the blunt end of the surgical tool 408 may include a pin at its tip to puncture through fascia. Additionally or alternatively, if the physical measurement of fascia indicates that the fascia is fully breached and the real-time images from the ultrasonic sensor 406 indicate the internal point of the target site has been reached by the surgical tool, the surgical tool 408 and the ultrasonic sensor 406 may stop moving.

FIG. 4B shows a block diagram 401 of the robotic surgical system 402 according to at least one embodiment of the present disclosure. In some examples, the robotic surgical system 402 may include a display screen 412 coupled to the ultrasonic sensor 406, where the display screen 412 receives and displays the real-time images from the ultrasonic sensor 406 of the surgical tool 408 relative to the target site. As shown, the ultrasonic sensor 406 and the display screen 412 may be coupled via a wire 414, but any type of connection can exist to transfer the real-time images generated by the ultrasonic sensor 406 to the display screen (e.g., wireless transmitter/receiver, Bluetooth connection, etc.). The real-time images may show layers of fascia 416 disposed between the outer layer of skin 410 and an internal point of the target site of the patient. In some examples, the real-time images of the surgical tool 408 relative to the target site received from the ultrasonic sensor 406 may include ultrasound images.

While the example described in FIGS. 4A and 4B discuss receiving and displaying images from the ultrasonic sensor 406, the ultrasonic sensor 406 may generate and transmit other types of data than image data for controlling movement of the first robot arm 404 and the ultrasonic sensor 406. That is, the robotic surgical system 402 may receive data other than image data from the ultrasonic sensor 406 and then control the surgical tool 408 (e.g., the tissue displacement instrument) based on that data. For example, the data generated and transmitted by the ultrasonic sensor 406 may be a different format than an image format (e.g., not organized according to pixels) or different than a static image (e.g., such as a video).

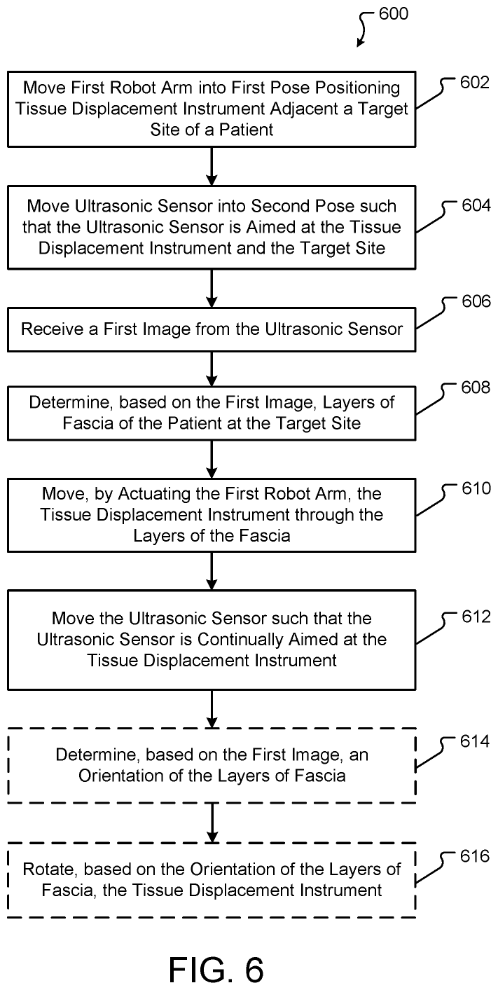

FIG. 5 depicts a method 500 that may be used, for example, to operate the robotic surgical system as described herein to synchronously move a first robot arm and an ultrasonic sensor together, where the first robot arm includes a tissue displacement instrument (e.g., surgical tool) capable of cutting and creating tissue pathways.

The method 500 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 500. The at least one processor may perform the method 500 by executing elements stored in a memory such as the memory 106. The elements stored in the memory and executed by the processor may cause the processor to execute one or more steps of a function as shown in method 500. One or more portions of a method 500 may be performed by the processor executing any of the contents of memory, such as an image processing 120, a segmentation 122, a transformation 124, and/or a registration 128.

The method 500 comprises moving the first robot arm into a first pose positioning the tissue displacement instrument adjacent a target site of a patient (step 502). In some examples, the target site of the patient may be a location on the surface of skin of the patient that corresponds to an internal point that is the target of the surgical operation or may be the internal point.

The method 500 also comprises moving the ultrasonic sensor into a second pose such that the ultrasonic sensor is aimed at the tissue displacement instrument and the target site, the ultrasonic sensor being coupled to the first robot arm via the at least one processor (step 504). By having the processor coupled to both the first robot arm and the ultrasonic sensor, the ultrasonic sensor may move or may be moved based on how the first robot arm moves to ensure that the ultrasonic sensor is continually aimed at the tissue displacement instrument and the target site. For example, a position of the ultrasonic sensor may be inferred from the detection of the tissue displacement instrument attached to the first robot arm using a reference coordinate system.

The method 500 also comprises receiving a first image from the ultrasonic sensor of the tissue displacement instrument relative to the target site (step 506). For example, the first image may be an ultrasound image that includes an image of internal areas of the patient.

The method 500 also comprises determining, based on the first image, layers of fascia of the patient at the target site, the layers of fascia being disposed between an outer layer of skin and an internal point of the target site (step 508). For example, the first image may indicate a presence of layers of fascia; a distance between the tissue displacement instrument and the layers of fascia; a measurement of the layers of fascia; whether the layers are unbreached, partially breached, or fully breached; etc. Accordingly, based on the first image and the determined layers of fascia, the first robot arm and the second robot arm may move (e.g., to continue the surgical operation).

The method 500 also comprises moving, by actuating the first robot arm, the tissue displacement instrument through the layers of the fascia while continuing to receive real-time images of the tissue displacement instrument relative to the target site (step 510). In some examples, moving the tissue displacement instrument through the layers of fascia may include using the blade end of the tissue displacement instrument to puncture/pierce/breach the layers of fascia. Additionally or alternatively, if the layers of fascia have been fully breached (e.g., as indicated by the first image), the tissue displacement instrument may move through the layers of fascia using the blunt end to create a tissue pathway. Additionally or alternatively, if the layers of fascia have been fully breached, the tissue displacement instrument may stop moving into the patient (e.g., if the internal point of the target site has been reached).

The method 500 also comprises moving the ultrasonic sensor such that the ultrasonic sensor is continually aimed at the tissue displacement instrument and the target site as the tissue displacement instrument is moved (step 512). By moving the second robot arm and the ultrasonic sensor to keep the ultrasonic sensor continually aimed at the tissue displacement instrument and the target site as the tissue displacement instrument is moved, the ultrasonic sensor can keep providing the real-time images of the tissue displacement instrument relative to the target site to ensure that the tissue displacement instrument moves through the patient safely.

The present disclosure encompasses embodiments of the method 500 that comprise more or fewer steps than those described above, and/or one or more steps that are different than the steps described above.

FIG. 6 depicts a method 600 that may be used, for example, to further operate the robotic surgical system as described herein to synchronously move a first robot arm and an ultrasonic sensor together, where the first robot arm includes a tissue displacement instrument (e.g., surgical tool) capable of cutting and creating tissue pathways

The method 600 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 600. The at least one processor may perform the method 600 by executing elements stored in a memory such as the memory 106. The elements stored in memory and executed by the processor may cause the processor to execute one or more steps of a function as shown in method 600. One or more portions of a method 600 may be performed by the processor executing any of the contents of memory, such as an image processing 120, a segmentation 122, a transformation 124, and/or a registration 128.

The method 600 comprises moving the first robot arm into a first pose positioning the tissue displacement instrument adjacent a target site of a patient (step 602). The method 600 also comprises moving the ultrasonic sensor into a second pose such that the ultrasonic sensor is aimed at the tissue displacement instrument and the target site, the ultrasonic sensor being coupled to the first robot arm via a processor (step 604). The method 600 also comprises receiving a first image from the ultrasonic sensor of the tissue displacement instrument relative to the target site (step 606). The method 600 also comprises determining, based on the first image, layers of fascia of the patient at the target site, the layers of fascia being disposed between an outer layer of skin and an internal point of the target site (step 608). The method 600 also comprises moving, by actuating the first robot arm, the tissue displacement instrument through the layers of the fascia while continuing to receive real-time images of the tissue displacement instrument relative to the target site (step 610). The method 600 also comprises moving the ultrasonic sensor such that the ultrasonic sensor is continually aimed at the tissue displacement instrument and the target site as the tissue displacement instrument is moved (step 612).

The method 600 also comprises determining, based on the first image, an orientation of the layers of fascia (step 614). The orientation of the layers of fascia may indicate a proximity of bones to the layers of fascia, a physical measurement of the layers of fascia (e.g., a thickness of the layers of fascia), other characteristics of the layers of fascia, or a combination thereof.

The method 600 also comprises rotating, based on the orientation of the layers of fascia, the tissue displacement instrument between a first tool position for cutting fascia to a second tool position displacing fascia (step 616). For example, the tissue displacement instrument may rotate between the first tool position for cutting fascia to the second tool position for displacing fascia based on whether the layers of fascia are breached or not, a proximity of the tissue displacement instrument to the layers of fascia and/or an internal point of the target site, etc. By having the tissue displacement instrument include both the blade end and the blunt end, a single tool can be used to reach the internal point of the target site without having to change out instruments or tools, thereby reducing the time needed to perform a corresponding surgical operation.

The present disclosure encompasses embodiments of the method 600 that comprise more or fewer steps than those described above, and/or one or more steps that are different than the steps described above.

As noted above, the present disclosure encompasses methods with fewer than all of the steps identified in FIGS. 5 and 6 (and the corresponding description of the methods 500 and 600), as well as methods that include additional steps beyond those identified in FIGS. 5 and 6 (and the corresponding description of the methods 500 and 600). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A robotic surgical system, comprising:
a first robot arm comprising a proximal end and a distal end;
a surgical tool attached to the distal end of the first robot arm, the surgical tool comprising a tissue displacement instrument;
an ultrasonic sensor;
a processor coupled with the first robot arm and the ultrasonic sensor; and
a memory coupled with and readable by the processor and storing therein data that, when executed by the processor, cause the processor to:
move the first robot arm into a first pose positioning the tissue displacement instrument adjacent a target site of a patient;
move the ultrasonic sensor into a second pose such that the ultrasonic sensor is aimed at the tissue displacement instrument and the target site;
receive a first image from the ultrasonic sensor of the tissue displacement instrument relative to the target site;
determine, based on the first image, layers of fascia of the patient at the target site, the layers of fascia being disposed between an outer layer of skin and an internal point of the target site;
move, by actuating the first robot arm, the tissue displacement instrument through the layers of the fascia while continuing to receive real-time images of the tissue displacement instrument relative to the target site; and
move the ultrasonic sensor such that the ultrasonic sensor is continually aimed at the tissue displacement instrument and the target site as the tissue displacement instrument is moved.

2. The robotic surgical system of claim 1, wherein the tissue displacement instrument comprises:
a first end comprising a blade; and
a second end comprising a blunt end, wherein the surgical tool is rotatable along an axis disposed between the first end and the second end, wherein, in a first tool position, the first end is disposed adjacent the target site, and wherein, in a second tool position, the surgical tool is rotated about the axis disposing the second end adjacent the target site in place of the first end.

3. The robotic surgical system of claim 2, wherein the data further cause the processor to:
determine, based on the first image, an orientation of the layers of fascia; and
rotate, based on the orientation of the layers of fascia, between the first tool position for cutting fascia to the second tool position for displacing fascia.

4. The robotic surgical system of claim 1, wherein the data further cause the processor to:
receive a second image from the ultrasonic sensor of the tissue displacement instrument as the tissue displacement instrument is moved; and
determine, based on the second image, one or more characteristics of the layers of fascia being disposed between the outer layer of skin and the internal point of the target site.

5. The robotic surgical system of claim 4, wherein the data further cause the processor to:
stop movement of the tissue displacement instrument and the ultrasonic sensor based on the one or more characteristics.

6. The robotic surgical system of claim 5, wherein the one or more characteristics comprise a physical measurement of the layers of fascia, an indication that the layers of fascia are fully breached, or a combination thereof.

7. The robotic surgical system of claim 4, wherein the data further cause the processor to:
move the tissue displacement instrument and the ultrasonic sensor based on the one or more characteristics.

8. The robotic surgical system of claim 7, wherein the data to move the tissue displacement instrument and the ultrasonic sensor further cause the processor to:
rotate the tissue displacement tool to a first tool position for cutting fascia from a second tool position for displacing fascia based on the one or more characteristics.

9. The robotic surgical system of claim 7, wherein the one or more characteristics comprise a physical measurement of the layers of fascia, an indication that one or more layers of the layers of fascia are yet to be breached, or a combination thereof.

10. The robotic surgical system of claim 1, further comprising:
a display screen coupled to the ultrasonic sensor, wherein the display screen receives and displays the real-time images from the ultrasonic sensor of the tissue displacement instrument relative to the target site.

11. The robotic surgical system of claim 10, wherein the real-time images received from the ultrasonic sensor of the tissue displacement instrument comprise ultrasound images.

12. The robotic surgical system of claim 1, wherein the ultrasonic sensor comprises a transducer.

13. The robotic surgical system of claim 1, further comprising:
a second robot arm comprising a proximal end and a distal end, wherein the ultrasonic sensor is attached to the distal end of the second robot arm.

14. A surgical robot, comprising:
a first robot arm comprising a proximal end and a distal end;
a surgical tool attached to the distal end of the first robot arm, the surgical tool comprising a tissue displacement instrument; and
an ultrasonic sensor, wherein the ultrasonic sensor is coupled to the first robot arm such that the ultrasonic sensor is continually aimed at the tissue displacement instrument and a target site as the tissue displacement instrument is moved.

15. The surgical robot of claim 14, wherein the tissue displacement instrument comprises:
a first end comprising a blade; and
a second end comprising a blunt end, wherein the surgical tool is rotatable along an axis disposed between the first end and the second end, wherein, in a first tool position, the first end is disposed adjacent the target site, and wherein, in a second tool position, the surgical tool is rotated about the axis disposing the second end adjacent the target site in place of the first end.

16. The surgical robot of claim 15, wherein the tissue displacement instrument is rotated between the first tool position for cutting fascia and the second tool position for displacing fascia based on a first image generated by the ultrasonic sensor.

17. The surgical robot of claim 16, wherein the first image generated by the ultrasonic sensor indicates an orientation of layers of fascia.

18. The surgical robot of claim 14, further comprising:
a display screen coupled to the ultrasonic sensor, wherein the display screen receives and displays real-time images from the ultrasonic sensor of the tissue displacement instrument relative to the target site.

19. The surgical robot of claim 18, wherein the real-time images from the ultrasonic sensor comprise ultrasound images.

20. A system, comprising:
a processor coupled with a first robot arm and an ultrasonic sensor; and
a memory coupled with and readable by the processor and storing therein data that, when executed by the processor, cause the processor to:
move the first robot arm into a first pose positioning a tissue displacement instrument adjacent a target site of a patient;
move the ultrasonic sensor into a second pose such that the ultrasonic sensor is aimed at the tissue displacement instrument and the target site;
receive a first image from the ultrasonic sensor of the tissue displacement instrument relative to the target site;
determine, based on the first image, layers of fascia of the patient at the target site, the layers of fascia being disposed between an outer layer of skin and an internal point of the target site;
move, by actuating the first robot arm, the tissue displacement instrument through the layers of the fascia while continuing to receive real-time images of the tissue displacement instrument relative to the target site; and
move the ultrasonic sensor such that the ultrasonic sensor is continually aimed at the tissue displacement instrument and the target site as the tissue displacement instrument is moved.
